(19) United States
(12) Patent Application Publication
(10) Pub. No.: US 2024/0344124 A1
(43) Pub. Date: Oct. 17, 2024

(54) Quantum Dot-Enrichment of CRISPR-Associated (Cas) Proteins for Environmental RNA Enrichment

(71) Applicant: The Government of the United States of America, as represented by the Secretary of the Navy, Arlington, VA (US)

(72) Inventors: Christopher M. Green, Washington, DC (US); Joseph R. Spangler, La Plata, MD (US); Tomasz A. Leski, Falls Church, VA (US); Kimihiro Susumu, Alexandria, VA (US); Sebastian A. Diaz, Silver Spring, MD (US); Igor L. Medintz, Springfield, VA (US); David A. Stenger, Annapolis, MD (US); Scott N. Dean, Washington, DC (US)

(21) Appl. No.: 18/509,355

(22) Filed: Nov. 15, 2023

Related U.S. Application Data
(60) Provisional application No. 63/495,604, filed on Apr. 12, 2023.

(51) Int. Cl. C12Q 1/6874; C12Q 1/6806

(52) U.S. Cl. CPC C12Q 1/6874; C12Q 1/6806; C12Q 2600/156

(57) ABSTRACT

One can enrich nucleic acid (RNA or DNA) from within a complex mixture in a sequence-specific manner by using a mutant of a Cas enzyme lacking nuclease activity, for example a mutant of Cas13a (dCas) from Leptotrichia wadeii. The nucleic acid is bound by the mutant protein as directed by guide RNA molecules (crRNA) and purified after association with an appropriate surface, such as quantum dots or functionalized beads. The nucleic acid is separated from the Cas and purified after precipitation or interaction with another surface. The resulting product can be directly processed via long-read sequencing.

This application claims the benefit of U.S. Provisional Patent Application No. 63/495,604 filed on Apr. 12, 2023, the entirety of which is incorporated herein by reference.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211333.

### INCORPORATION BY REFERENCE

This Application incorporates by reference the Sequence Listing XML file submitted herewith via the patent office electronic filing system having the file name “211333US2.xml” and created on Nov. 14, 2023 with a file size of 8,781 bytes.

### BACKGROUND

Nucleic acid sequencing has become increasingly important for pathogen identification and target detection in environmental samples. Current methods of nucleic acid detection have reached single-molecule resolution based on nucleic-acid directed targeting methods, but these techniques destroy the detected nucleic acid.

A need exists for techniques to enrich for specifically sought-after sequences, especially from within a complex mixture where the target is only present in miniscule amounts. This would enable the use of current generation sequencing technologies to generate the required coverage for positive identifications and typing without the need for excessive sample amounts.

### BRIEF SUMMARY

As described herein, one can concentrate long sequences of ribonucleic acid (RNA) from within a complex mixture (for example, one containing proteins and/or nucleic acids apart from a sequence of interest) in a sequence-specific manner. This involves the use of a form of the Cas protein that has been mutated in order to remove nuclease activity, for example Cas13a protein (dCas) from Leptotrichia wadeii, or dCas12, or dCas9. The RNA is bound by the mutant protein as directed by guide RNA molecules (crRNA) and purified after association with a particles configured to bind to the Cas protein, for example quantum dots (QDs) having a ZnS coating. The resulting product can be directly processed via RNA long-read sequencing.

In one embodiment, a method of concentrating a ribonucleic acid (RNA) includes providing a Cas13 protein mutant lacking RNA-hydrolysis activity and having a polyhistidine tag; contacting the Cas13 protein with a guide RNA (crRNA), a sample comprising a target RNA, and a quantum dot (QD), wherein the crRNA is capable of binding to both the Cas13 protein and to the target RNA; allowing the Cas13 protein and crRNA bind to target RNA in the sample to form a complex and allowing the QD nanoparticle binds to the complex to form a QD-complex; isolating the QD-complex; and digesting the Cas13 protein in the QD-complex with a protease to release the target RNA.

In another embodiment, a method of concentrating a nucleic acid includes providing a Cas protein mutant lacking nucleic-acid-hydrolysis activity and having a polyhistidine tag; contacting the Cas protein with a guide RNA (crRNA), a sample comprising a target nucleic acid, and a first surface functionalized to bind to the Cas protein, wherein the crRNA is capable of binding to both the Cas protein and to the target nucleic acid; allowing the Cas protein and crRNA bind to target nucleic acid in the sample to form a complex and allowing the first surface to bind to the complex to form a bound complex; rinsing the bound complex; digesting the Cas13 protein in the bound complex with a protease to release the target nucleic acid; allowing the released target nucleic acid to bind to a second surface; rinsing the target nucleic acid while bound to the second surface; and eluting the nucleic acid from the second surface. In various further embodiments, the first surface comprises agarose beads and said second surface comprises a filter; or the first surface comprises a first type of magnetic bead and said second surface comprises a second type of magnetic bead.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides a description of CRISPR-Cas functionality, uses, and applications. CRISPR-Cas systems are commonly used for multiple applications, with the proteins of interest and their associated activities outlined in the scheme. The advantages to CRISPR-Cas detection over traditional PCR-based detection methods are outlined based on minimal sequence requirements for identification and the literature-demonstrated limits of detection in the single molecule range. The use of mutated Cas13 enzymes that have altered HEPN (higher eukaryotes and prokaryotes nucleotide binding) domains, the collateral nucleic acid cleavage activity is removed, therefore allowing the pulldown of a target RNA based on the sequence of the guide (crRNA).

FIG. 2 illustrates one exemplary enrichment scheme as described herein, wherein a selected RNA is enriched for sequencing. The flowchart indicates the order of reagent addition and general molecular interactions that facilitate enrichment of the targeted RNA species as determined by crRNA complementarity. The mutated Cas13 protein is adsorbed to the quantum dot (QD625), where it becomes active by the addition of the crRNA, allowing it to bind the target RNA. The subsequent changes in pH drive the QD into an aggregated form allowing for washing and removing of non-target RNA species, and the addition of protease destroys the mutated Cas13 protein thereby releasing target and guide RNAs. These recovered RNAs can be subjected to manufacturer-specified preparation for sequencing and ultimately identified via the Oxford Nanopore MinION Mk1C platform.

FIGS. 3A and 3B depict the effect of the target enrichment protocol of FIG. 2 on direct RNA sequencing. FIG. 3A provides results of early experiments mixing a target RNA species (981 nt) with background total RNA from humans showed the differences in read abundance of the target RNA (purple) when comparing to a sample where all RNA was subjected to sequencing (yellow). Experiments involved diluting in vitro transcribed target RNA 50-fold or 100-fold (purple and yellow, respectively) into human total RNA by mass and carrying out the Oxford Nanopore Technologies (ONT) Direct RNA Sequencing Protocol, where either the entire RNA pool (yellow) or only the target RNA (purple) was subjected to polyadenylation sequencing. While still possible to obtain reads on the sequence-of-interest without selection or enrichment, the experiment outlines advantages of removing the background RNA from the sequencing protocol. FIG. 3B shows results from subsequent experiments with the enrichment protocol (yellow) show enhanced abundance of reads of the longer target RNAs over experiments without enrichment at all. This enrichment protocol included the dilution of in vitro transcribed target RNA 20-fold into total bacterial RNA by mass, and subjecting this RNA pool to the above-described enrichment protocol. These results are compared to controls without enrichment (purple) that are described in panel A (yellow). The output read lengths shown in both panels represent all polyadenylated RNA present in the samples. As the polyadenylation procedure is part of commercial sequencing preparation kits, there is an unspecified amount of adenosines added resulting in an increased size distribution of the target RNA. Additionally, sequencing without enrichment allows for natively polyadenylated background messenger RNAs to be sequenced. Therefore the processing of a total, unenriched RNA pool using the manufacturer's sequencing preparation protocol causes all RNAs to be polyadenylated and sequenced, but in comparison the introduction of an upstream enrichment protocol selects sequence-specific target RNAs only results in their selective sequencing regardless of sample complexity or nucleic acid background.

FIGS. 4A and 4B depict the effect of enrichment as described in FIG. 2 on PCR-cDNA sequencing. Enrichment methods were proven to capable of retaining target RNA with increasing sample complexity whether a short target RNA was used (˜200 nt, A) or a longer target RNA was used (981 nt, B). The PCR-cDNA sequencing protocol is similar to the Direct RNA sequencing protocol shown above with the exception of an additional polymerase chain reaction step to increase nucleic acid abundance before the sequencing reaction. As these samples undergo polyadenylation, the size distribution of target RNAs increases as a result of sequencing preparation. The experiments were carried out by diluting in vitro transcribed RNA into either tRNA (FIG. 4A) or bacterial total RNA (FIG. 4B) at the specified percentages by mass. Each sample was subjected to the above-described enrichment protocol and subsequently processed by the manufacturer's specified protocol for PCR-cDNA sequencing. The two major peaks in FIG. 4A represent the guide RNA from the enrichment protocol at smaller length, and the target RNA that only results from enrichment protocol at longer lengths. When analyzing sequencing runs aimed at enriching longer RNAs, the number of comparative reads for the long target are maintained despite increases in complexity (50% target RNA in total RNA background compared to 5% target RNA in total RNA background), indicating that the amount of target RNA captured is not a function of complexity, but instead is a function of the binding capabilities of the mutated Cas13 protein. These experiments prove that the target RNA enrichment protocol can be successful with both short and long RNA fragments based on sequence specificity in a variety of nucleic acid backgrounds of different origins, and are similarly compatible with multiple commercially available sequencing preparation protocols.

FIGS. 5A-5D depict a further embodiment of Cas-mediated enrichment employing centrifugation and Ni-NTA-agarose beads.

FIGS. 6A-6C provide results of targeted enrichment on sequencing depth of 981 nt lcrV target diluted in contaminant human RNA to (FIG. 6A) 10%, (FIG. 6B) 1%, and (FIG. 6C) 0.1% of the total mass of RNA. The total mass of RNA used was 250 ng for each experiment, and samples were sequenced using ONT's direct RNA sequencing protocol.

FIGS. 7A-7D relate to multiplexed enrichment. FIG. 7A illustrates the multiplexed enrichment of targets using a single enrichment mixture containing several distinct Cas13/crRNA complexes. FIGS. 7B-7D show the effects of multiplexed enrichment on direct RNA sequencing on samples consisting of: (b) 7249 nt M13mp18 RNA target diluted in contaminant total human RNA to 10% (FIG. 7B); 10165 nt lambda-10k RNA target diluted in contaminant total human RNA to 10% (FIG. 7C); 20432 nt lambda-20k RNA target diluted in contaminant total human RNA to 10% (FIG. 7D). The total mass of RNA used was 1000 ng for each experiment, and samples were sequenced using ONT's direct RNA sequencing protocol. Control samples were prepared at equivalent volume and concentration, then purified and concentrated with Zymo RNA clean and concentrator kits prior to preparation for sequencing.

FIGS. 8A-8C schematically depict the use of magnetic beads for this technique.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### OVERVIEW

Described herein is a novel technique to capture and enrich long sequences of nucleic acids (RNA or DNA) in a sequence-specific manner. Namely, the enrichment method uses novel nucleic-acid-binding protein mutants that bind specific sequences of interest, and enrich them from complicated matrices for subsequent purification and use with modern sequencing technology. Existing technologies capture DNA chemically or enzymatically destroy the native DNA during processing (to reduce the length of the nucleic acid down to manageable sizes for the old generation sequencing), or they capture RNA based on adherence to a poly(A) stretch (not sequence-specific) and similarly reduce the length of the nucleic acid for old generation sequencing.

One example of modern sequencing technology is the “third generation” sequencing technology involving long-read nanopores, namely that marketed by the company Oxford Nanopore Technologies (ONT). The biochemistry utilized allows for reads that are one to two orders of magnitude longer on average compared with previous technology. The longer reads come at the cost of significantly higher read errors, but these errors can be overcome by more sequencing depth (i.e., repetition). This requires a larger source sample of the target sequence, which can be a problem. This can involve an excessively large sample in the common situation when the target sequence is a small or even miniscule fraction of a complex matrix of other nucleotides, as is the case with many samples of biological origin.

As with sequencing, similar advancements have occurred in the past decade in systems utilizing CRISPR (clustered regularly interspaced short palindromic repeats), centering around the use of CRISPR-Associated proteins (Cas) and their ability to bind specific nucleic acid sequences that is guided by crispr RNA (crRNA, also termed guide RNA). There are many CRISPR-Cas systems, each with their own efficiencies, associated activities, and interaction specificities with certain structured crRNAs.

The present technique is based on a Cas13a protein that maintains (1) a simple crRNA design scheme and (2) serves to bind target RNA and hydrolyze RNA external to the protein:RNA complex. The Cas13a protein cloned from the organism Leptotrichia wadeii (LwCas13a) was mutated in two or four locations to remove any RNA-hydrolysis activity, thereby creating a protein that would specifically bind target RNA based on the directions given by our additionally designed crRNA, which is added to and bound by the Cas and is responsible for specifying the target RNA to be bound. The mutated protein (referred to as deactivated Cas13, or dCas13) was expressed with a hexahistidine (His₆) motif to facilitate the protein's initial purification during production. This motif also allows the protein to bind the ZnS surface of ZnS-overcoated core/shell semiconductor quantum dots (QDs) where the core is typically CdSe. The proteins bind via metal-affinity coordination between the imidazole functional group of the protein's histidine residues and the Zn on the QD surface as the latter is not an oxide. Displaying modified proteins on such QDs merely requires mixing them together in stoichiometric amounts, as the proteins self-assemble to the QDs almost instantaneously with high affinity. Protein packing on the QD surface is determined by QD and protein size, and follows a Poisson progression until a steric maxima is reached. Once formed, the resulting complex of QD-Cas-RNA can be gently removed from solution with changes in salinity and pH, i.e. by perturbing its colloidal stability, therefore allowing the purification of the specifically captured RNA from the complex matrix. After subsequent washing, the Cas protein is hydrolyzed from the QD using proteolytic enzymes, and the released RNA can be concentrated and used directly for ONT sequencing.

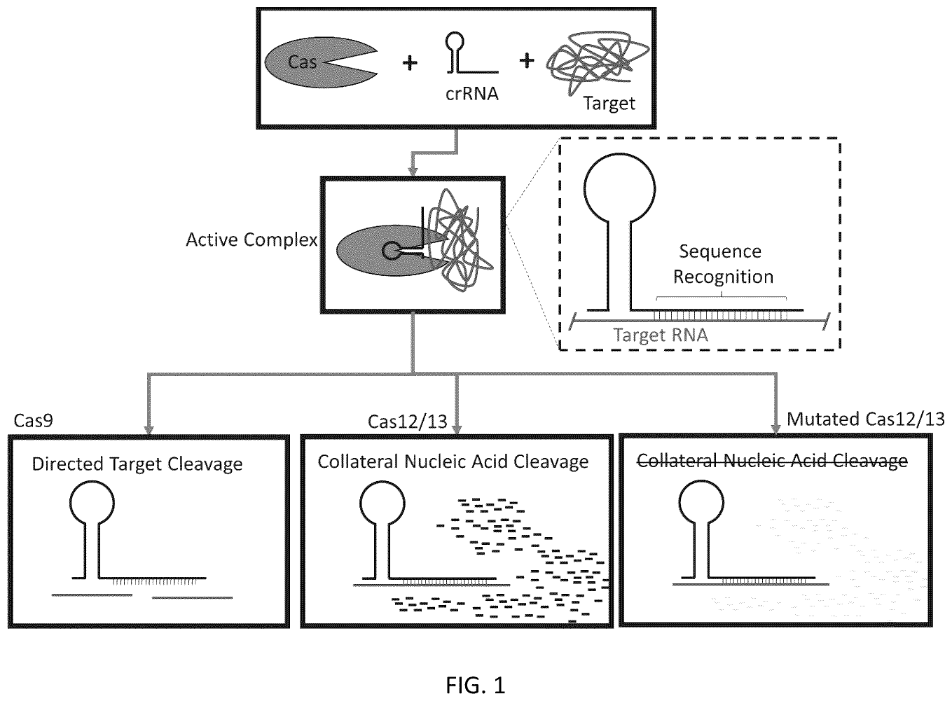

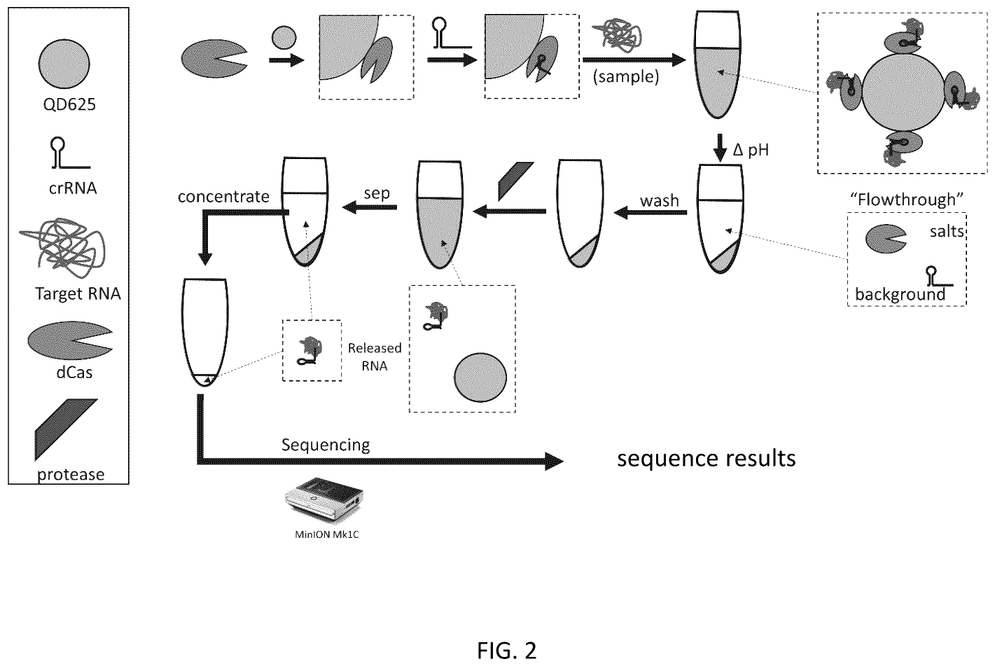

This technology allows for sequence-specific enrichment of ribonucleic acid molecules from various matrices via binding, sedimentation, and release of the designed complex for subsequent processing for sequence analysis. Guide RNAs (crRNAs) are designed complementary to target sequences-of-interests (FIG. 1), and are then combined with the deactivated mutant LwCas13a to form a ternary complex, after which the complex is conjugated to the surface of ZnS overcoated QDs and pulled out of solution by changes in pH and ionic strength to perturb its colloidal stability followed by centrifugal force (FIG. 2). The complex matrix of unwanted nucleic acid and other molecules is removed, and the isolated complex is washed and released from QDs by protease digestion. The released RNA in solution is separated from the QDs, and can be further concentrated to prepare for sequencing analysis following established manufacturer protocols.

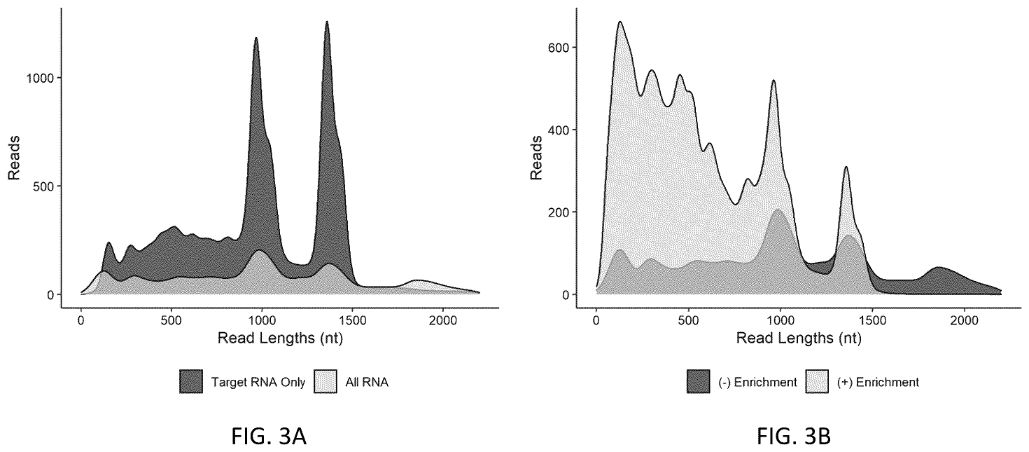

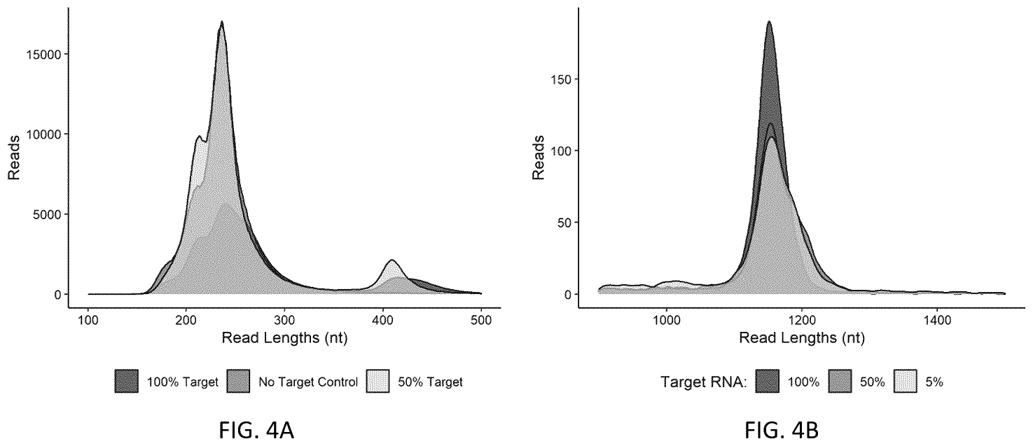

Guide RNAs can be designed so that specific nucleic acid architecture is maintained along with planned mismatches in otherwise complementary (spacer) regions to increase binding efficiency of target RNA molecule. The crRNA lengths are typically 67 nt in total, with 28 nt devoted to complementarity to the target RNA molecule to be bound by dCas13. The components of the reaction (crRNA, dCas13, target RNA/sample, QD) are combined stepwise and allowed to assemble at 37° C. in a low salt buffer (50 mM Tris, pH 8, 6 mM NaCl, 10 mM MgCl₂) to maintain stability of all components. After formation of ternary Cas:RNA:RNA complex adsorbed to QD surface, solution pH is lowered by the addition of sodium acetate and QD-complex is pelleted by gentle centrifugal force. Unbound sample matrix (non-targeted RNAs) is then physically removed, and upon multiple washes of QD-Cas complex the captured RNA is released through digestion of the Cas protein by protease, and the now-enriched RNA is prepared for sequencing via ONT MinION platform. The enrichment scheme allows for the targeted sequencing of specified long RNAs despite increasing amounts of sample complexity (FIGS. 3A, 3B, 4A, and 4B), and the amount of RNA resulting from the enrichment protocol can be controlled by the amount of Cas protein used.

An effective stoichiometric molar ratio of the dCas:crRNA:target:QD components was found to be 10:100:1:1.

### EXEMPLARY METHODS

Reagents. All reagents unless otherwise specified were obtained from Sigma Aldrich.

Mutated LwaCas13. Mutated LwaC2c2 was generated by GenScript (Piscataway, NJ) with the specific mutations R474A and R1046A (double mutant) and R474A, H479A, R1046A and H1051A (quad mutant).

RNA Preparation. Guide RNA (cr7) and target RNA were prepared as described in Spangler et al., 2022 (incorporated herein by reference for the purposes of teaching such techniques) by in vitro transcription using HiScribe T7 Quick High Yield RNA Synthesis Kits (New England Biolabs, Ipswich, MA) using template DNA oligonucleotides from Integrated DNA Technologies (Coralville, IA). These target RNAs consisted of the full length lcrV RNA (981 nt) from Y pestis strain C092 (Schultzhaus et al., 2021) and the shortened lcrV RNA (237 nt) (Spangler et al., 2022). The 27 nt target RNA labeled with Alexa488 was synthesized by Integrated DNA Technologies. All sequences are given in Table 1

Quantum Dots. Quantum Dots were prepared as described in Susumu et al., 2007. Briefly, QD625 was acquired from Sigma Aldrich (Qdot 625 ITK carboxyl) and ligand exchanged with compact zwitterionic ligands composed of a dihydrolipoic acid anchor group and terminating in two alkyl carboxyl groups.

Enrichment. RNA enrichments were set up using initial binding assays as a guideline with molar ratios of 10:100:1:1 (dCas:crRNA:QD:target RNA) with 2 pmol target RNA to insure target RNA was the limiting reagent. Reactions occurred in 200 μL volumes where QD was combined with dCas in buffer (50 mM Tris, pH 8, 0.6 M NaCl, 0.01 M MgCl₂) and incubated for 10 min at 37° C. Guide RNA was added to reactions and then incubated another 10 min at 37° C., followed by addition of the target RNA pre-diluted in background and a final incubation for 30 min at 37° C. Following these incubation steps, samples were supplemented 100 μL 3M sodium acetate (pH 5.2) to begin disrupting QD colloidal stability, and pelleted gently for 5 min at 6,000×g. Pellet was washed twice after supernatant removal with 300 μL volumes of buffer (0.3 M sodium acetate, pH 5.2, 0.6 M NaCl, 0.01 M MgCl₂) followed by gentle pelleting and supernatant removal again. Pellet was resuspended in 300 μL buffer (50 mM Tris, pH 7.5, 0.006 M NaCl, 0.01 M MgCl₂) followed by the addition of 0.8 U proteinase K (New England Biolabs) and incubation for 1 h at 37° C. After proteinase digestion of dCas13, samples were gently pelleted, 300 μL supernatant was kept supplemented with 30 μL 3 M sodium acetate (pH 5.2), 10 μg glycogen and 1 mL ethanol for ethanol precipitation at −20° C. for 30 min. Subsequent pellets were resuspended in 15 μL nuclease free water and added to 2 μL 10×PolyA polymerase buffer, 2 μL 10 mM ATP, and 1 μL polyA polymerase (NEB) and incubated 15 min at 37° C. following the manufacturer's protocol. Following adenylation, samples were ethanol precipitated again and resuspended in 10 μL nuclease free water for quantification and sequencing library preparation.

Sequencing Library Preparation. High throughput sequencing of the baseline and enriched RNA target preparations was performed using MinION Mk 1C sequencer (Oxford Nanopore Technologies PLC, Oxford, UK). In order to prepare RNA libraries for sequencing, the polyA tails were added using E. coli Poly(A) Polymerase (New England Biolabs, Ipswich, MA) according to the manufacturer's instructions, using 30 min incubation with 5U of the enzyme. The RNA was purified using Clean & Concentrator 25 kit (Zymo research, Irvine, CA). The purified RNA was quantified using Qubit fluorometer with RNA High Sensitivity reagent kit (Thermo Fisher, Grand Island, NY) and used as input for the Nanopore sequencing library preparation protocols. Three distinct library preparation workflows were used utilizing the following Oxford Nanopore reagent kits: direct RNA sequencing kit (SQK-RNA002), cDNA-PCR sequencing kits (SQK-PCS109) and cDNA-PCR sequencing kit with barcoding (SQK-PCB109). Library preparation was conducted according to the manufacturer instructions except with lower input RNA quantities (approximately 10×, at a range of 6 to 60 ng adapted RNA) than the recommended input of 200 ng. The libraries were loaded into Spot-ON flow cell (version R9, FLO-MIN106D) and run for 72 hours using default settings. Sequencing runs using Direct RNA protocol were carried out individually, whereas those with PCR-cDNA were run in multiplex.

Analysis. Sequence alignments were carried out on random samples of 10,000 pass reads that were further derived from subsets of read widths described in individual experiments. Unless otherwise noted, the workflow was as follows. After binned reads were selected, all RNA sequences from were converted to DNA for alignment using a nucleotide substitution matrix (match=1, mismatches=−3 with the option to only recognize the 4 canonical bases). Pairwise alignments were carried out using the overlap strategy with addition penalties of 5 for gap openings and 2 for gap extensions, and the aligned sequences were cutoff below scoring thresholds based on experimental details for further analysis. In general, longer reads on full length lcrV gene were subjected to alignment cutoff scores of 300 and 100 for short reads. Similarly, long and short reads were cutoff below scores of 100 for experiments using a shortened lcrV reference. Reads being aligned to guide RNA were also cutoff below scores of 30 with a gap extension penalty of 0. The percentage of mapped reads for Direct RNA experiments was calculated as the number of reads passing alignment scoring criteria over the sample population, and the total mapped reads were estimated by applying this map percentage to the total number of reads passing initial quality filters from the sequencer. PCR-cDNA protocol results were calculated similarly, with the exception of percentages being the sum of alignments to both reference and reverse complement over the sample population considering these protocols generate dsDNA. Target RNA coverage was determined by evaluating the individual results of pairwise alignments and extracting the metadata related to the initial aligned nucleotide position (Start) relative to the reference sequence and the width of the aligned read (Width), and visually mapping each aligned read with respect to its position along the reference sequence. Analysis was carried out using the ShortRead and BioStrings packages in R.

### Advantages

The technology offers several advantages to prior art. Complex RNA extractions from unknown samples (organisms, environmental samples, etc.) can be probed to isolate specific intact RNAs amenable to commercial sequencing protocols while maintaining the fidelity to facilitate the state-of-the-art long read sequencing such as that provided by Oxford Nanopore Technology. Furthermore, using the dCas13 protein allows RNA to be specifically selected without needing to previously fractionate RNA from DNA, increasing the target RNA scope to include RNA viruses.

Moreover, the sequence-specific enrichment of RNA molecules allows for up to millions of reads directed towards enriched RNAs instead of total sample.

The enrichment of long RNA molecules is suited to current sequencing technology. Previous techniques for nucleic acid enrichment require fragmentation to smaller (300 nt) fragments, as these methods were developed for the previous generation of sequencing technology. Cas9-enrichment schemes exist capable of pulling out DNA only, and the resulting DNAs are short fragments as a result of Cas9-directed cleavage.

Using the dCas13 protein provides for the non-destructive analysis of a sample, such that samples can be re-analyzed indefinitely for either iterative enrichments or reach-back analysis at later dates. This contrasts with alternative enrichment methods which are destructive. As described herein, samples can be enriched for one specific target, and enrichment can be repeated with different crRNAs to enrich for additional targets in the same sample.

Furthermore, this technique involves ambient temperature-stable capture: all components maintain desirable activity at room temperature given longer incubation times.

It also provides the ability to visually track capture protocol. Photo-luminescent properties of QDs allow for visual inspection with excitation under ultraviolet (UV) light.

The required reagents are water-based, and steps are carried out without the use of hazardous chemicals.

QDs provide multiple characteristics/properties to enable further optimization and to provide other utility to this technique. The QDs can also host a molecular beacon type sensor on its surface to signal binding events in real-time by FRET where the QD is the FRET donor or the molecular beacon has both the donor and acceptor incorporated into it. The degree of display of Cas proteins can be controlled by stoichiometric ratios that can contribute to a higher localized binding affinity and a higher probability of a capture event. Different colors/sizes of QDs can be mixed together with each hosting different Cas proteins for multiplexed enrichment schemes. Differential precipitation can help isolate different QD complexes sequentially. The QD component can be lyophilized and then rehydrated and reconstituted when needed, therefore facilitating long-term storage. The QD has the potential to host other proteins on its surface as bound in a similar manner. This can allow for other functionalities. The QD can host Cas proteins with different crRNAs targeting the same strand of target RNA at different areas, therefore allowing for higher affinity capture of rarer targets by multipoint interactions.

### FURTHER EMBODIMENTS

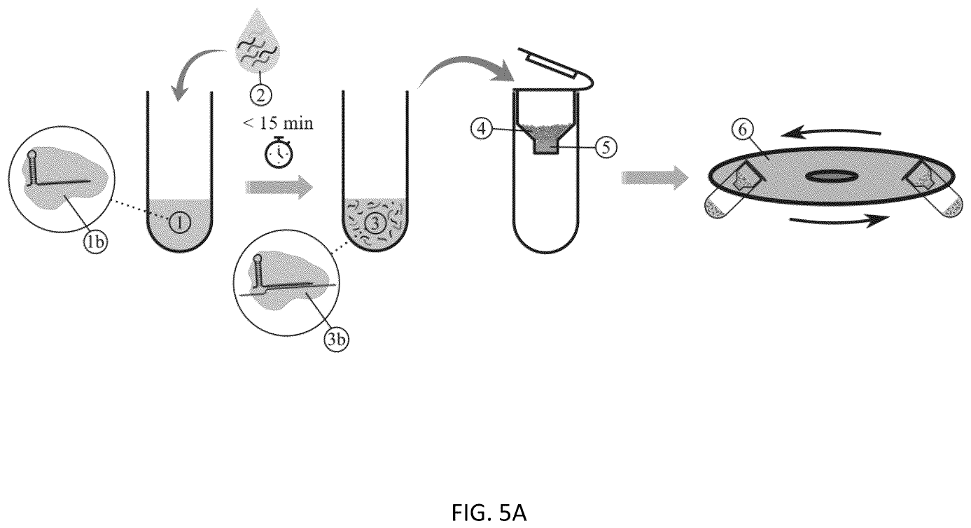

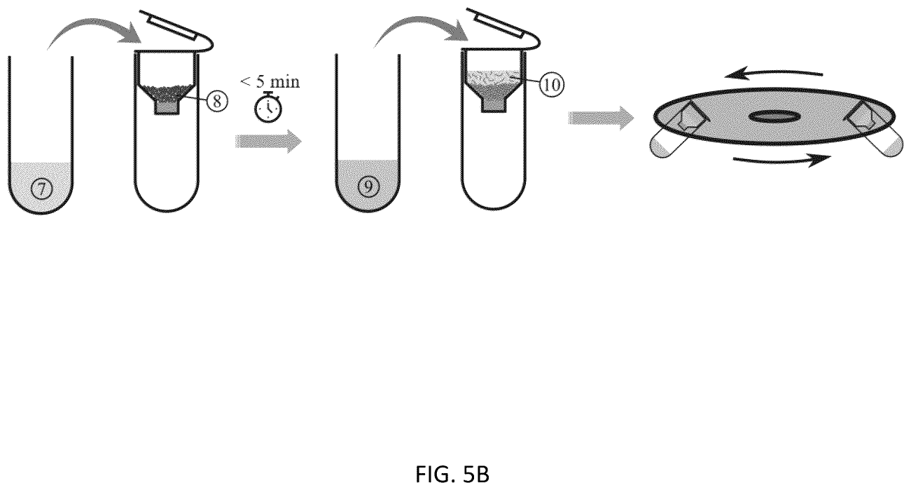

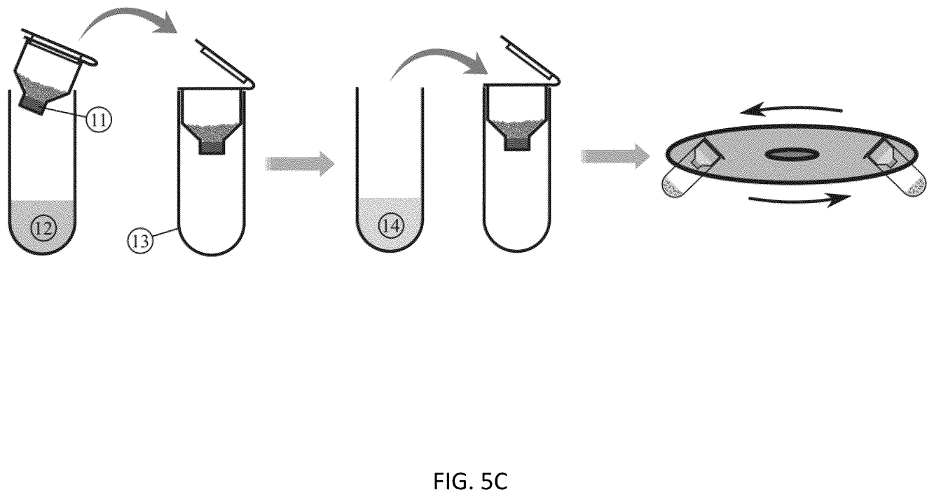

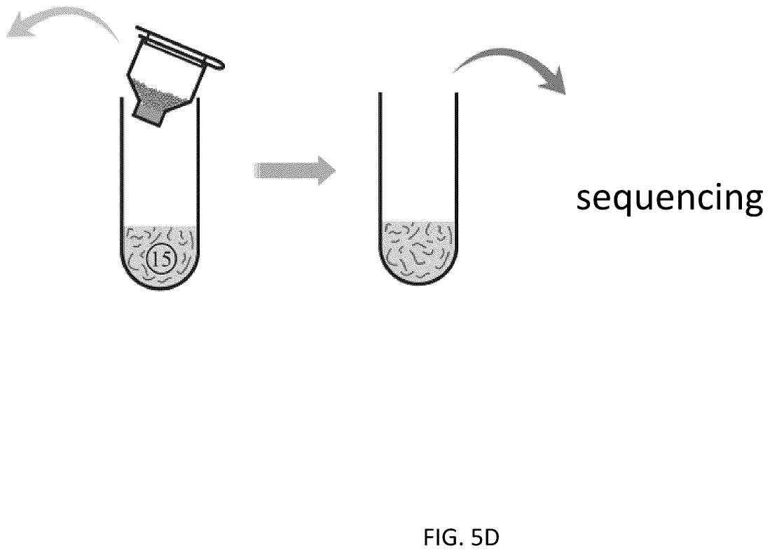

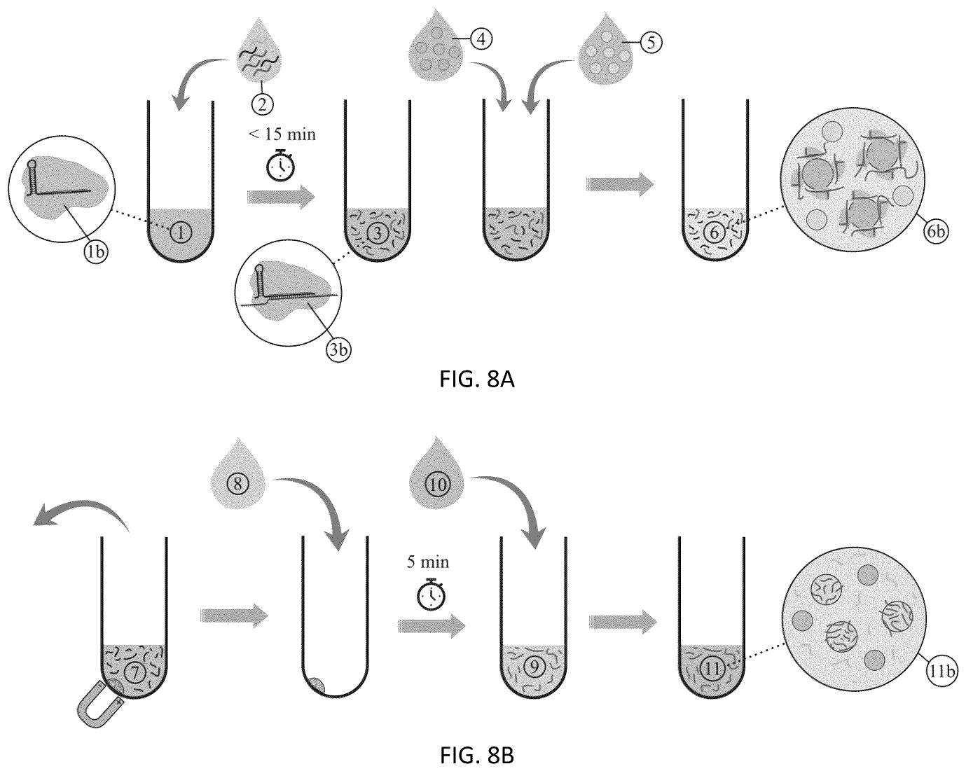

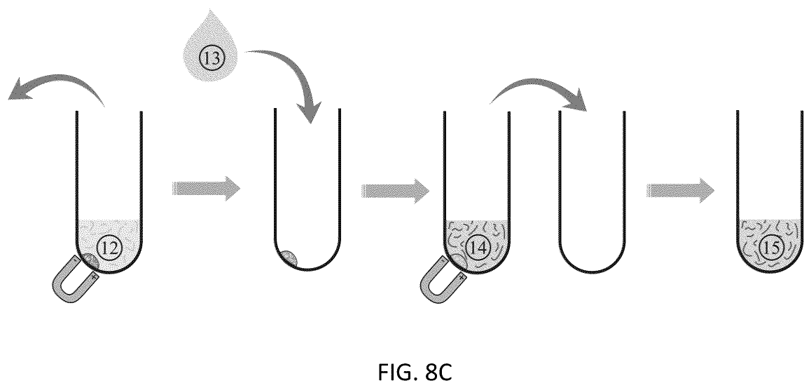

It is also possible to employ a Cas-based enrichment technique using capture of Cas proteins bound to target RNA on a suitable surface such as a functionalized microparticle, as depicted in FIG. 5 (in conjunction with column acting as a second surface to collect released target), or FIG. 8 with magnetic capture (using one type of bead to capture the Cas complex from the sample and another type to capture the released target). In certain embodiments, the entire enrichment process can take place within a single vessel.

In particular, the capture and enrichment of target RNA and/or DNA can be accomplished in a single pot using a combination of CRISPR technology with bead- and column-based pulldown and purification. Deactivated Cas proteins (dCas13, dCas12, or dCas9), preassembled with specific guide RNAs, are employed to identify and capture desired RNA and/or DNA from a complex sample mixture, after which they are transferred to a centrifuge tube containing agarose beads functionalized with ligands such as Ni-NTA, streptavidin, or carboxyl ligands. These beads or other colloidal microparticles facilitate the capture of Cas proteins bound to target nucleic acids. Subsequent centrifugation at low speeds allows for the removal of supernatant, and the captured nucleic acid-Cas complex could easily be subjected to several buffer rinse cycles to ensure purity. To release the target nucleic acid from the complex, buffers containing ample quantities of EDTA (and/or other chelators) and proteinases are introduced to digest the Cas proteins. The composition of a binding buffer combined with ethanol is then adjusted for nucleic acid binding with the possibility of size-based selection. After another round of separation (e.g., centrifugation), the selectively bound nucleic acid targets remains in a filter (such as a syringe filter or continuous flow filter, or a spin column for a microcentrifuge) or on a second functionalized bead or microparticle, while contaminants are removed in the flow-through. To improve the level of purification, additional rinse steps can be performed. The final, purified nucleic acid can be transferred to a new, nuclease-free vessel (e.g., microcentrifuge tube) with a minimal volume of water or buffer by centrifugation, typically no less than 6 μL.

This provides an amplification-free strategy for targeted RNA and DNA enrichment within RNA and DNA purification and concentration columns. This strategy enables highly efficient enrichment of full-length RNA transcripts, as well as selective separation of ssDNA, dsDNA, and RNA.

The rapid, targeted capture of RNA and/or DNA is achieved with nuclease-deactivated CRISPR-associated (Cas) proteins, for example dCas13. Further contemplated embodiments employ other Cas-family enzymes, Cas-fusion enzymes, or target-selective nucleic acid binding proteins such as Fanzor proteins, argonaute proteins, transcription activator-like effectors, zinc finger nucleases, REX proteins, and/or meganucleases to achieve the targeted capture.

Highly multiplexed enrichment can be achieved in a single, diverse mixture of Cas/crRNA pairs which distinguish targets with distinct spacer sequences. Massively multiplexed enrichment and/or high-throughput screening can be achieved by application of this method in microplate format.

As described in the below example, agarose beads can serve as a surface for the selective binding and pulldown of Cas enzymes. Other embodiments could utilize nanoparticles and/or microparticles for pulldown, such as metallic nanoparticles, magnetic nanoparticles, hybrid nanoparticles, carbon-based nanoparticles, polymer beads, silica-based nanoparticles, and/or quantum dots.

Cas enzymes displaying polyhistidine tags were selectively bound by Ni-NTA groups on the agarose beads to facilitate pulldown. Further embodiments could facilitate binding of protein to nanoparticles and/or microparticles by biotin-streptavidin binding, EDC/NHC chemistry, maleimide-thiol chemistry, click chemistry, silane coupling, and/or thiol-metal binding.

Separation of bound Cas/target on beads from contaminant RNA and DNA was achieved by centrifugation. The unbound fraction passes through the column and is discarded, while Cas/target complexes stay bound to the agarose beads and remain within the column. Other contemplated embodiments include separation in a syringe-based column format, magnetic separation, pH-induced pulldown, ionic strength-induced pulldown, ligand-induced colloidal instability, and/or crosslinking-based aggregation.

Release of target RNA and/or DNA from Cas was achieved with proteinase digestion and/or high EDTA buffers. Other contemplated embodiments include light or radiation-induced release, thermally driven release, addition of denaturants, pH changes, cleavage by proteases, and targeted protein degradation methods.

Target RNA and/or DNA was purified from protein digest and other contaminants and concentrated using RNA and/or DNA purification and concentration columns. In the embodiment depicted in FIG. 5, the full enrichment process was performed in a Zymo-spin column. RNA and/or DNA purification and concentration was achieved with RNA clean & concentrator chemicals from Zymo Research Corporation. It is expected that the technique could also be accomplished with embodiments such as DNA and/or RNA purification kits and columns from QIAGEN, Promega, Thermo Fisher Scientific, Ambion, Norgen Biotek, New England Biolabs, Cytiva, and Purigen Biosystems. It would also be possible for a person skilled in the art to develop their own reagents and equipment to perform the described methods.

Target RNA and/or DNA was selectively bound in the column filter by the addition of RNA binding buffer and ethanol prior to centrifugation. Target remains bound within the column while protein digests and other contaminants are removed in the flow-through. Enriched and concentrated target was then eluted into a new nuclease free tube with a small volume of nuclease-free water or buffer for downstream processing and analyses.

This technique provides the highly efficient enrichment of RNA and/or DNA transcripts in an amplification-free format, with little to no loss of native target in highly diluted samples and little if any loss in integrity of native targets. It significantly reduces sequencing bandwidth lost to contaminant nucleic acids. Furthermore, it does not require PCR for amplification, though it is compatible with downstream amplification if desired.

The sequence-specific enrichment of full-length transcripts is achieved without use of primers or cDNA. This enables native RNA sequencing of full-length, enriched RNA transcripts and enriches only RNA targets specified by guide RNA spacer sequence. Furthermore, it does not require the design and/or use of complex cDNA or primers for PCR.

Furthermore, highly multiplexed enrichment is possible with use of multiplexed guide RNAs. Multiplexed guides can be used in a single mixture for multiplexed capture of targets. Guide RNA design for multiplexed targets is computationally simple in comparison to cDNA and PCR primer design for multiplexed targets. Multiplexing can also be performed in series as the flow-through from target pulldown is not compromised in integrity.

This technique can be performed in a single column filter to minimize material requirements and cost, or in a multi-well microplate to maximize throughput and multiplexing capacity. Agarose bead capture of protein/target complexes and all subsequent steps can be performed in a single nucleic acid purification and concentration column. Filter columns with agarose beads can be prepared and shipped after drying by centrifugation and are compatible with storage at ambient conditions.

The capture and pulldown are not specific to Cas enzymes; other DNA and/or RNA binding protein that displays a polyhistidine tag can undergo pulldown and be used for enrichment with this technique.

The technique offers the additional advantages of using only water and ethanol-based reagents, and all reactions can be performed at room temperature and ambient conditions. The protein capture of target and subsequent digestion of protein and release of target can be accelerated at elevated temperatures but is not required. Polyhistidine-based capture of proteins is driven by self-assembly and does not require special reagents or chemical reactions.

Potential exists for significantly greater multiplexing bandwidth than cDNA and PCR-based strategies. The strategic use of DNA and/or RNA binding proteins with greater or lesser sequence specificity can be employed to capture broad families of viral and/or bacterial target genomes, and/or to capture specific mutations of genomes within a specific family. Guides can be designed to target highly conserved regions of a genome and/or highly variable regions depending on the intended application.

The following provides an illustrative example using RNA, however it could be extended to DNA by substituting Cas12 or Cas9 for Cas13, as long as the Cas protein lacks nucleic-acid-hydrolysis activity (for example, by the introduction of an appropriate mutation).

As shown in FIG. 5A, an extracted RNA sample containing target RNA and complex background of contaminant RNA 2 is added to mixture of preassembled dCas13a/crRNA 1 (inset with figure reference 1b refers to dCas13a/crRNA ternary complex) to form the Cas/sample mixture 3. The inset with figure reference 3b depicts dCas13a/crRNA/target complex. The Cas complex binds the target, typically within 15 minutes, then the Cas/target mixture is transferred to a DNA/RNA purification column 5 containing pre-rinsed Ni-NTA-agarose beads 4. Polyhistidine tags on the Cas selectively bind to the beads, and the solution is centrifuged briefly 6 to remove contaminant RNA and buffer. Preferably, several additional rinses are performed to improve purity.

In FIGS. 5B and 5C, Cas digestion buffer 7, containing proteinases and a high EDTA concentration, is added to the column containing Ni-NTA-agarose beads with bound Cas/target 8 and incubated for 5 min to release target. Manufacturer-provided RNA binding solution 9 is added to the digested Cas mixture, and the solution is centrifuged briefly. Target RNA 10 is bound in the column 11 filter while protein digests, buffer, and other contaminants are collected in the flow-through 12. Preferably, several brief rinsing steps follow to ensure target purity. Then, the column with captured target 11 is transferred to a clean tube 13 (free of nucleases), then a small volume of pure water or buffer 14 is added to the column. The sample is centrifuged to elute the target 15.

The column can be removed and stored for later use as in FIG. 5D, and the enriched target mixture 15 is prepared for sequencing with nanopore sequencing (for example, ONT from Oxford Nanopore Technologies) or other techniques.

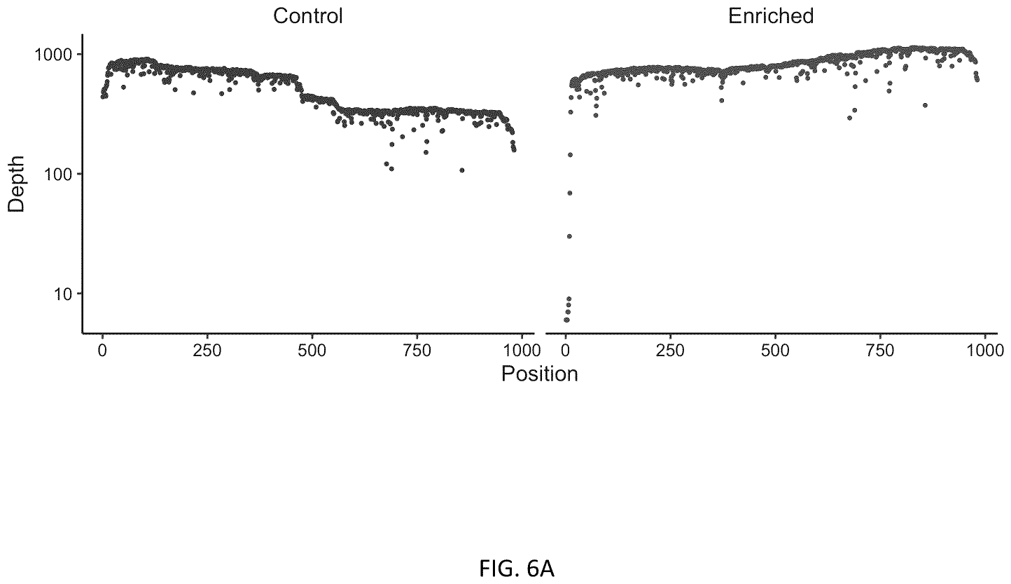

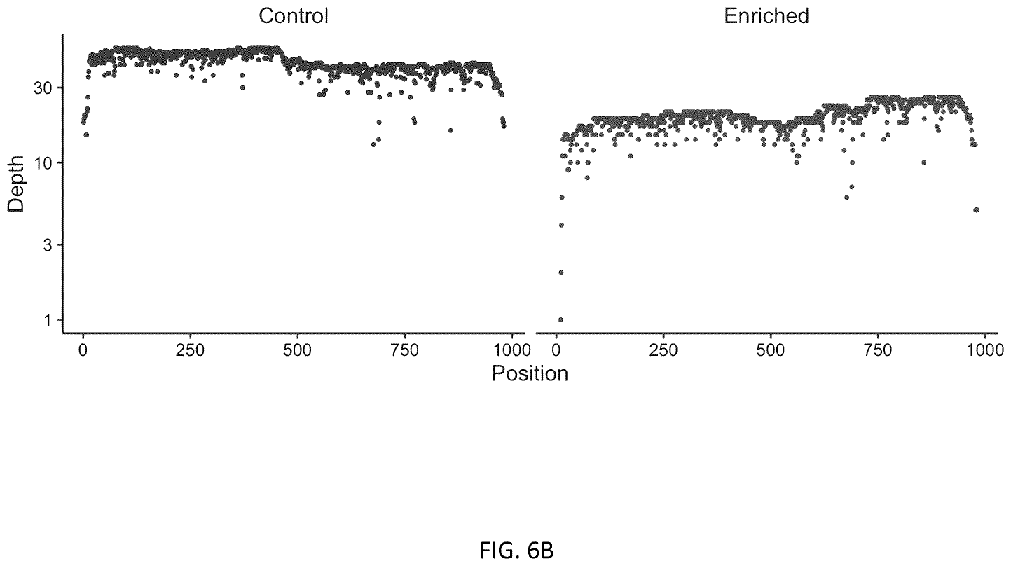

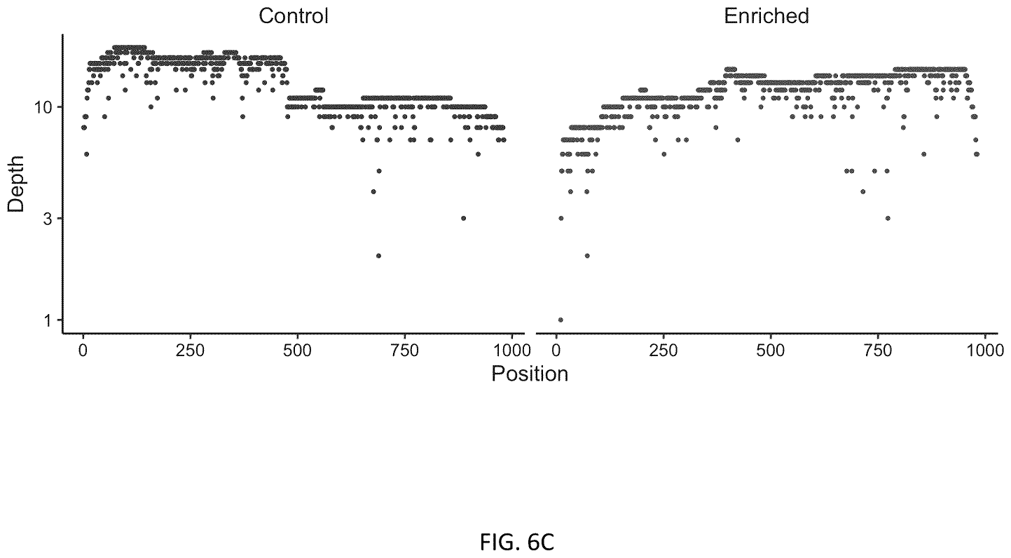

FIGS. 6A-6C show results of targeted enrichment on sequencing depth of 981 nt lcrV target diluted in contaminant human RNA to 10%, 1%, and 0.1% of the total mass of RNA, using this protocol with agarose beads. The total mass of RNA used was 250 ng for each experiment, and samples were sequenced using ONT's direct RNA sequencing protocol. The sequencing results for a positive control (lcrV only, 100.0%) and negative control (total human RNA, 0.0%) are also provided. The technique demonstrated up to 47-fold enrichment of target in the first demonstrated experiment. The sequencing results for a positive control (lcrV only) and negative control (total human RNA) are not shown but are provided in Table 2.

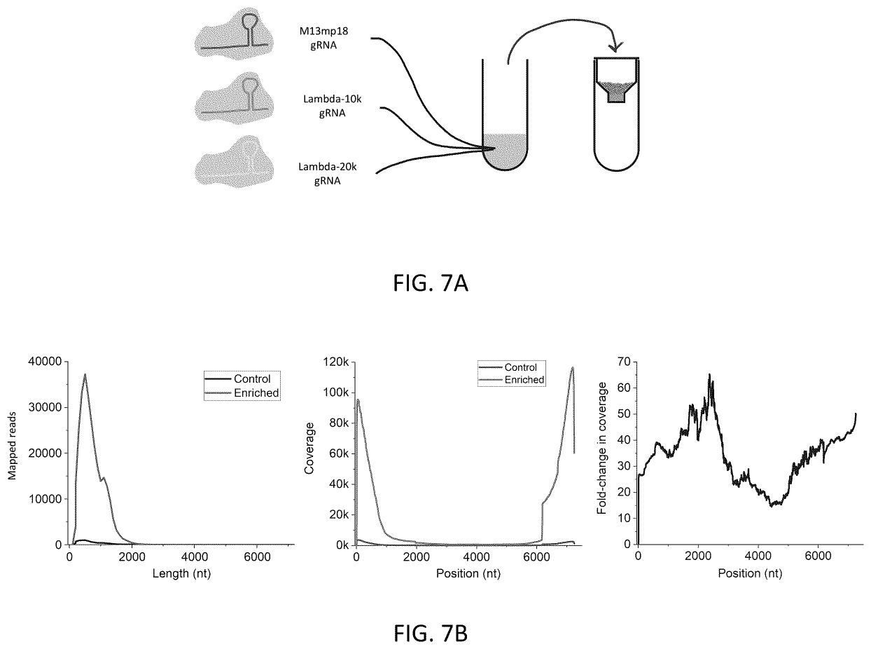

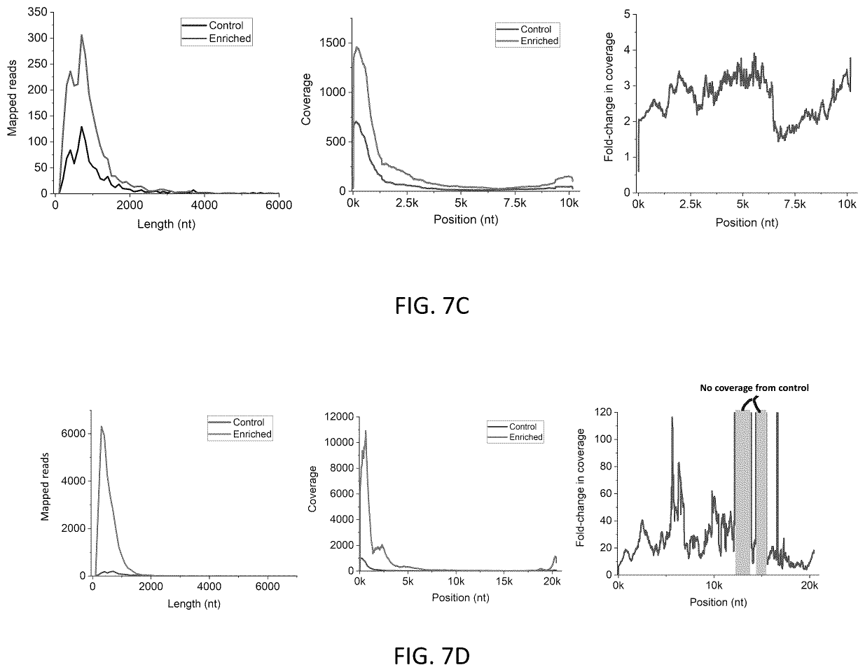

FIGS. 7A-7D and Table 3 provide data relating to multiplexed target enrichment on direct RNA sequencing of three large RNA transcripts in complex background, again using the protocol described above with agarose beads. Each transcript was diluted to 10% (w/w) in total human RNA, and control samples were prepared directly for sequencing while enriched samples were prepared for sequencing using multiplexed target enrichment. For all transcripts, the enrichment protocol produced greater total reads, mapped reads, and % mapped reads than direct sequencing of the RNA mixtures. For samples with optimized crRNA design (M13mp18 and lambda-20k), the enrichment protocol resulted in greater than 20-fold increase in the number of mapped reads and 10-fold increase in the percentage of reads mapped to target.

Magnetic beads (including magnetic nanoparticles) with distinct surface chemistries for binding proteins and/or for binding nucleic acids could be used simultaneously in a manner analogous to the described combination of protein-binding beads and nucleic acid-binding column filter. Examples include Ni-NTA-magnetic beads, and Zymo RNA clean & concentrator magnetic beads. Mixing these beads together could provide sequential Cas-capture on Ni-NTA-magnetic beads, followed by digestion of Cas and release of target, then addition of RNA binding buffer to bind RNA to the Zymo beads, and protein digests would be separated out, the Zymo beads with RNA would be rinsed, and RNA would be released from the Zymo beads in a purified and concentrated form. This could all occur in a single tube.

FIGS. 8A-8C schematically illustrate a prophetic exemplary enrichment process involving magnetic beads. First, in FIG. 8A, an extracted RNA sample containing target nucleic acids and complex background of contaminant nucleic acids 2, is added to mixture of preassembled dCas/crRNA 1 (the inset with reference 1b refers to dCas13a/crRNA ternary complex) to form the Cas/sample mixture 3. The inset with reference 3b depicts dCas13a/crRNA/target complex. The Cas complex binds the target, typically within 15 min, then a combination of both protein-binding 4 and DNA/RNA-binding 5 magnetic beads are added to the sample, thus creating a magnetic bead/Cas/sample mixture 6. The Cas/target complexes selectively bind to the protein-binding magnetic beads as seen in the inset at 6b. Then, as seen in FIG. 8B, the solution is purified of non-target nucleic acids by pulldown 7 of the magnetic beads and their bound Cas/target, and supernatant containing non-target is discarded. Several additional rinses are performed to improve purity. Cas digestion buffer 8, containing proteinases and a high EDTA concentration, is added to the magnetic beads and incubated for 5 min to release target 9. DNA/RNA binding solution 10 is added to the digested Cas mixture to produce a Cas digest with magnetic beads and bound target 11, and released target nucleic acids bind to the DNA/RNA-binding magnetic beads (inset 11b). In FIG. 8C, target nucleic acids are bound to the magnetic beads 12 while protein digests, buffer, and other contaminants are collected in the supernatant. Several brief rinsing steps with follow to ensure target purity. Nuclease-free water or buffer 13 is added to the magnetic beads to release target nucleic acids. The magnetic beads are pulled down 14, and the supernatant containing enriched and purified sample can be transferred to a new nuclease-free tube 15 and then, if desired, subjected to sequencing.

TABLE 1
Nucleotide and amino acid sequences

| Sequence Name | Sequence (5′ to 3′ or N to C) |
|---|---|
| cr7 | GGGGAUUUAGACUACCCCAAAAACGAAGGGGACUAAAACAAUA |
| (SEQ ID NO: 1) | UCUAUAUUUUUAUCUUUGACUAAC |
| lcrV target | AUGAUUAGAGCCUACGAACAAAACCCACAACAUUUUAUUGAGG |
| (SEQ ID NO: 2) | AUCUAGAAAAAGUUAGGGUGGAACAACUUACUGGUCAUGGUUC |
| | UUCAGUUUUAGAAGAAUUGGUUCAGUUAGUCAAAGAUAAAAAU |
| | AUAGAUAUUUCCAUUAAAUAUGAUCCCAGAAAAGAUUCGGAGG |
| | UUUUUGCCAAUAGAGUAAUUACUGAUGAUAUCGAAUUGCUCAA |
| | GAAAAUCCUAGCUUAUUUUCUACCCGAGGAUGCCAUUCUUAAA |
| | GGCGGUCAUUAUGACAACCAACUGCAAAAUGGCAUCAAGCGAG |
| | UAAAAGAGUUCCUUGAAUCAUCGCCGAAUACACAAUGGGAAUU |
| | GCGGGCGUUCAUGGCAGUAAUGCAUUUCUCUUUAACCGCCGAU |
| | CGUAUCGAUGAUGAUAUUUUGAAAGUGAUUGUUGAUUCAAUGA |
| | AUCAUCAUGGUGAUGCCCGUAGCAAGUUGCGUGAAGAAUUAGC |
| | UGAGCUUACCGCCGAAUUAAAGAUUUAUUCAGUUAUUCAAGCC |
| | GAAAUUAAUAAGCAUCUGUCUAGUAGUGGCACCAUAAAUAUCC |
| | AUGAUAAAUCCAUUAAUCUCAUGGAUAAAAAUUUAUAUGGUUA |
| | UACAGAUGAAGAGAUUUUUAAAGCCAGCGCAGAGUACAAAAUU |
| | CUCGAGAAAAUGCCUCAAACCACCAUUCAGGUGGAUGGGAGCG |
| | AGAAAAAAAUAGUCUCGAUAAAGGACUUUCUUGGAAGUGAGAA |
| | UAAAAGAACCGGGGCGUUGGGUAAUCUGAAAAACUCAUACUCU |
| | UAUAAUAAAGAUAAUAAUGAAUUAUCUCACUUUGCCACCACCU |
| | GCUCGGAUAAGUCCAGGCCGCUCAACGACUUGGUUAGCCAAAA |
| | AACAACUCAGCUGUCUGAUAUUACAUCACGUUUUAAUUCAGCU |
| | AUUGAAGCACUGAACCGUUUCAUUCAGAAAUAUGAUUCAGUGA |
| | UGCAACGUCUGCUAGAUGACACGUCUGGUAAAUGA |
| shortened lcrV | AUUGAGGAUCUAGAAAAAGUUAGGGUGGAACAACUUACUGGUC |
| target | AUGGUUCUUCAGUUUUAGAAGAAUUGGUUCAGUUAGUCAAAGA |
| (SEQ ID NO: 3) | UAAAAAUAUAGAUAUUUCCAUUAAAUAUGAUCCCAGAAAAGAU |
| | UCGGAGGUUUUUGCCAAUAGAGUAAUUACUGAUGAUAUCGAAU |
| | UGCUCAAGAAAAUCCUAGCUUAUUUUCUACCCGAGGAUGCCAU |
| | UCUUAAAGGCGGUCAUUAUGAC |
| Lwa C2c2 | MKVTKVDGISHKKYIEEGKLVKSTSEENRTSERLSELLSIRLD |
| (dCas13) double | IYIKNPDNASEEENRIRRENLKKFFSNKVLHLKDSVLYLKNRK |
| mutant | EKNAVQDKNYSEEDISEYDLKNKNSFSVLKKILLNEDVNSEEL |
| (SEQ ID NO: 4) | EIFRKDVEAKLNKINSLKYSFEENKANYQKINENNVEKVGGKS |
| | KRNIIYDYYRESAKRNDYINNVQEAFDKLYKKEDIEKLFFLIE |
| | NSKKHEKYKIREYYHKIIGRKNDKENFAKIIYEEIQNVNNIKE |
| | LIEKIPDMSELKKSQVFYKYYLDKEELNDKNIKYAFCHFVEIE |
| | MSQLLKNYVYKRLSNISNDKIKRIFEYQNLKKLIENKLLNKLD |
| | TYVRNCGKYNYYLQVGEIATSDFIARNRQNEAFLRNIIGVSSV |
| | AYFSLRNILETENENDITGRMRGKTVKNNKGEEKYVSGEVDKI |
| | YNENKQNEVKENLKMFYSYDFNMDNKNEIEDFFANIDEAISSI |
| | AHGIVHFNLELEGKDIFAFKNIAPSEISKKMFQNEINEKKLKL |
| | KIFKQLNSANVFNYYEKDVIIKYLKNTKFNFVNKNIPFVPSFT |
| | KLYNKIEDLRNTLKFFWSVPKDKEEKDAQIYLLKNIYYGEFLN |
| | KFVKNSKVFFKITNEVIKINKQRNQKTGHYKYQKFENIEKTVP |
| | VEYLAIIQSREMINNQDKEEKNTYIDFIQQIFLKGFIDYLNKN |
| | NLKYIESNNNNDNNDIFSKIKIKKDNKEKYDKILKNYEKHNRN |
| | KEIPHEINEFVREIKLGKILKYTENLNMFYLILKLLNHKELTN |
| | LKGSLEKYQSANKEETFSDELELINLLNLDNNRVTEDFELEAN |
| | EIGKFLDFNENKIKDRKELKKFDTNKIYFDGENIIKHRAFYNI |
| | KKYGMLNLLEKIADKAKYKISLKELKEYSNKKNEIEKNYTMQQ |
| | NLHRKYARPKKDEKFNDEDYKEYEKAIGNIQKYTHLKNKVEFN |
| | ELNLLQGLLLKILHRLVGYTSIWERDLRFRLKGEFPENHYIEE |
| | IFNFDNSKNVKYKSGQIVEKYINFYKELYKDNVEKRSIYSDKK |
| | VKKLKQEKKDLYIANYIAHFNYIPHAEISLLEVLENLRKLLSY |
| | DRKLKNAIMKSIVDILKEYGFVATFKIGADKKIEIQTLESEKI |
| | VHLKNLKKKKLMTDRNSEELCELVKVMFEYKALEHHHHHH |
| Lwa C2c2 | MKVTKVDGISHKKYIEEGKLVKSTSEENRTSERLSELLSIRLD |
| (dCas13) quad | IYIKNPDNASEEENRIRRENLKKFFSNKVLHLKDSVLYLKNRK |
| mutant | EKNAVQDKNYSEEDISEYDLKNKNSFSVLKKILLNEDVNSEEL |
| (SEQ ID NO: 5) | EIFRKDVEAKLNKINSLKYSFEENKANYQKINENNVEKVGGKS |
| | KRNIIYDYYRESAKRNDYINNVQEAFDKLYKKEDIEKLFFLIE |
| | NSKKHEKYKIREYYHKIIGRKNDKENFAKIIYEEIQNVNNIKE |
| | LIEKIPDMSELKKSQVFYKYYLDKEELNDKNIKYAFCHFVEIE |
| | MSQLLKNYVYKRLSNISNDKIKRIFEYQNLKKLIENKLLNKLD |
| | TYVRNCGKYNYYLQVGEIATSDFIARNRQNEAFLRNIIGVSSV |
| | AYFSLRNILETENENDITGRMRGKTVKNNKGEEKYVSGEVDKI |
| | YNENKQNEVKENLKMFYSYDFNMDNKNEIEDFFANIDEAISSI |
| | AHGIVAFNLELEGKDIFAFKNIAPSEISKKMFQNEINEKKLKL |
| | KIFKQLNSANVFNYYEKDVIIKYLKNTKFNFVNKNIPFVPSFT |
| | KLYNKIEDLRNTLKFFWSVPKDKEEKDAQIYLLKNIYYGEFLN |
| | KFVKNSKVFFKITNEVIKINKQRNQKTGHYKYQKFENIEKTVP |
| | VEYLAIIQSREMINNQDKEEKNTYIDFIQQIFLKGFIDYLNKN |
| | NLKYIESNNNNDNNDIFSKIKIKKDNKEKYDKILKNYEKHNRN |
| | KEIPHEINEFVREIKLGKILKYTENLNMFYLILKLLNHKELTN |
| | LKGSLEKYQSANKEETFSDELELINLLNLDNNRVTEDFELEAN |
| | EIGKFLDFNENKIKDRKELKKFDTNKIYFDGENIIKHRAFYNI |
| | KKYGMLNLLEKIADKAKYKISLKELKEYSNKKNEIEKNYTMQQ |
| | NLHRKYARPKKDEKFNDEDYKEYEKAIGNIQKYTHLKNKVEFN |
| | ELNLLQGLLLKILHRLVGYTSIWERDLRFRLKGEFPENHYIEE |
| | IFNFDNSKNVKYKSGQIVEKYINFYKELYKDNVEKRSIYSDKK |
| | VKKLKQEKKDLYIANYIAAFNYIPHAEISLLEVLENLRKLLSY |
| | DRKLKNAIMKSIVDILKEYGFVATFKIGADKKIEIQTLESEKI |
| | VHLKNLKKKKLMTDRNSEELCELVKVMFEYKALEHHHHHH |

TABLE 2
Results of targeted enrichment on sequencing depth of 981 nt lcrV target diluted
in contaminant human RNA to 10%, 1%, and 0.1% of the total mass of RNA.

| | Control samples | | Enriched | | |
|---|---|---|---|---|---|
| Target lcrV | | Percent mapped | | Percent mapped | Enrichment |
| concentration | Total reads | 750 nt-1500 nt fraction | Total reads | 750 nt-1500 nt fraction | (fold) |
| 10.0% | 194636 | 0.352 | 18045 | 16.600 | 47.2 |
| 1.0% | 45585 | 0.176 | 1849 | 5.240 | 29.8 |
| 0.1% | 213393 | 0.010 | 23146 | 0.268 | 27.9 |
| 100.0% | 42645 | 46.58 | — | — | — |
| 0.0% | 565184 | 0 | — | — | — |

TABLE 3
Results of multiplexed target enrichment on direct RNA sequencing
of three large RNA transcripts in complex background.

| | Control samples | | | Enriched | | |
|---|---|---|---|---|---|---|
| | Total | Mapped | % | Total | Mapped | % |
| Target | reads | reads | mapped | reads | reads | mapped |
| 10% M13mp18 | 221778 | 6263 | 2.8 | 815162 | 211123 | 25.9 |
| 10% lambda- | 331905 | 947 | 0.29 | 731977 | 2533 | 0.35 |
| 10k | | | | | | |
| 10% lambda- | 264000 | 1483 | 0.64 | 491997 | 31434 | 6.39 |
| 20k | | | | | | |

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

Each of the below documents is incorporated herein by reference for the purposes of disclosing methods and materials relating to the technological area in which it is cited.

### Nucleic Acid Enrichment for Sequencing

Gabrieli, T., et al. (2018). “Selective nanopore sequencing of human BRCA1 by Cas9-assisted targeting of chromosome segments (CATCH).” Nucleic Acids Res 46(14): e87.
Gilpatrick, T., et al. (2020). “Targeted nanopore sequencing with Cas9-guided adapter ligation.” Nat Biotechnol 38(4): 433-438.
Liu, X., et al. (2017). “In Situ Capture of Chromatin Interactions by Biotinylated dCas9.” Cell 170(5): 1028-1043 e1019.
McDonald, T. L., et al. (2021). “Cas9 targeted enrichment of mobile elements using nanopore sequencing.” Nat Commun 12(1): 3586.
Metsky, H. C., et al. (2019). “Capturing sequence diversity in metagenomes with comprehensive and scalable probe design.” Nat Biotechnol 37(2): 160-168.
Mighell, T. L., et al. (2022). “Cas12a-Capture: A Novel, Low-Cost, and Scalable Method for Targeted Sequencing.” The CRISPR Journal5(4): 548-557.
Schultzhaus, Z., et al. (2021). “CRISPR-based enrichment strategies for targeted sequencing.” Biotechnol Adv 46: 107672.
Slesarev, A., et al. (2019). “CRISPR/CAS9 targeted CAPTURE of mammalian genomic regions for characterization by NGS.” Sci Rep 9(1): 3587.
Stangl, C., et al. (2020). “Partner independent fusion gene detection by multiplexed CRISPR-Cas9 enrichment and long read nanopore sequencing.” Nat Commun 11(1): 2861.
Watson, C. M., et al. (2020). “Cas9-based enrichment and single-molecule sequencing for precise characterization of genomic duplications.” Lab Invest 100(1): 135-146.
Xu, X., et al. (2019). “CRISPR-assisted targeted enrichment-sequencing (CATE-seq).” BioRxiv: 672816.

### CRISPR/Cas Biosensing

Abudayyeh, O. O., et al. (2017). “RNA targeting with CRISPR-Cas13.” Nature 550(7675): 280-284.
Abudayyeh, O. O., et al. (2016). “C2c2 is a single-component programmable RNA-guided RNA-targeting CRISPR effector.” Science 353(6299): aaf5573.
Ackerman, C. M., et al. (2020). “Massively multiplexed nucleic acid detection with Cas13.” Nature 582(7811): 277-282.
Barnes, K. G., et al. (2020). “Deployable CRISPR-Cas13a diagnostic tools to detect and report Ebola and Lassa virus cases in real-time.” Nature communications 11(1): 1-10.
Kellner, M. J., et al. (2019). “SHERLOCK: nucleic acid detection with CRISPR nucleases.” NatureProtocols 14(10): 2986-3012.
Metsky, H. C., et al. (2020). “CRISPR-based surveillance for COVID-19 using genomically-comprehensive machine learning design.” BioRxiv.
Myhrvold, C., et al. (2018). “Field-deployable viral diagnostics using CRISPR-Cas13.” Science 360(6387): 444-448.
Sohail, M., et al. (2022). “Molecular reporters for CRISPR/Cas: From design principles to engineering for bioanalytical and diagnostic applications.” TrAC Trends in Analytical Chemistry 149: 116539.
Spangler et al. (2022). “Large scale screening of CRISPR guide RNAs using an optimized high throughput robotics system” Sci. Rep. 2022 Aug. 17; 12(1):13953.

### Metal Affinity Coordination of Enzymes and Other Proteins to ODs

Delehanty, J. B., et al. (2006). “Self-assembled quantum dot-peptide bioconjugates for selective intracellular delivery.” Bioconjugate Chemistry 17(4): 920-927.
Medintz, I. L., et al. (2004). “A fluorescence resonance energy transfer-derived structure of a quantum dot-protein bioconjugate nanoassembly.” Proc NatlAcad Sci USA 101(26): 9612-9617.
Medintz, I. L., et al. (2005). “Quantum dot bioconjugates for imaging, labelling and sensing.” Nature Materials 4(6): 435-446.
Noh, M., et al. (2010). “Fluorescence quenching caused by aggregation of water-soluble CdSe quantum dots.” Colloids and Surfaces A: Physicochemical and Engineering Aspects 359(1-3): 39-44.
Susumu, K., et al. (2007). “Enhancing the stability and biological functionalities of quantum dots via compact multifunctional ligands.” Journal of the American Chemical Society 129(45): 13987-13996.

What is claimed is:

1. A method of concentrating a ribonucleic acid (RNA), the method comprising:
providing a Cas13 protein mutant lacking RNA-hydrolysis activity and having a polyhistidine tag;
contacting the Cas13 protein with a guide RNA (crRNA), a sample comprising a target RNA, and a quantum dot (QD), wherein the crRNA operates to bind both the Cas13 protein and the target RNA;
allowing the Cas13 protein and crRNA bind to target RNA in the sample to form a complex and allowing the QD nanoparticle to bind to the complex to form a QD-complex;
isolating the QD-complex; and
digesting the Cas13 protein in the QD-complex with a protease to release the target RNA.

2. The method of claim 1, further comprising sequencing the target RNA after the release thereof.

3. The method of claim 1, wherein the active complex is allowed to form prior to contacting with said QD.

4. The method of claim 1, wherein said Cas13 protein has an amino acid sequence selected from the group consisting of SEQ ID NO: 4 and SEQ ID NO: 5.

5. A method of concentrating a nucleic acid, the method comprising:
providing a Cas protein mutant lacking nucleic-acid-hydrolysis activity and having a polyhistidine tag;
contacting the Cas protein with a guide RNA (crRNA), a sample comprising a target nucleic acid, and a first surface functionalized to bind to the Cas protein, wherein the crRNA is capable of binding to both the Cas protein and to the target nucleic acid;
allowing the Cas protein and crRNA bind to target nucleic acid in the sample to form a complex and allowing the first surface to bind to the complex to form a bound complex;
rinsing the bound complex;
digesting the Cas protein in the bound complex with a protease to release the target nucleic acid;
allowing the released target nucleic acid to bind to a second surface;
rinsing the target nucleic acid while bound to the second surface; and
eluting the nucleic acid from the second surface.

6. The method of claim 5, wherein said first surface comprises agarose beads and said second surface comprises a filter configured to bind to nucleic acid.

7. The method of claim 5, wherein said first surface comprises a first type of magnetic bead and said second surface comprises a second type of magnetic bead.

8. The method of claim 5, further comprising sequencing the target nucleic acid after said elution.

9. The method of claim 5, wherein said Cas protein has an amino acid sequence selected from the group consisting of SEQ ID NO: 4 and SEQ ID NO: 5.


SEQUENCE LISTING (5 sequences)

SEQ ID NO: 1 - RNA, 67 nt, synthetic construct
  source 1..67: mol_type = other RNA
ggggatttagactaccccaaaaacgaaggggactaaaacaatatctatatttttatctttgactaac

SEQ ID NO: 2 - RNA, 981 nt, synthetic construct
  source 1..981: mol_type = other RNA
atgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaatga

SEQ ID NO: 3 - RNA, 237 nt, synthetic construct
  source 1..237: mol_type = other RNA
attgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgac

SEQ ID NO: 4 - protein, 1158 aa, synthetic construct
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIAHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIANYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEHHHHHH

SEQ ID NO: 5 - protein, 1158 aa, synthetic construct
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIAHGIVAFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIANYIAAFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALEHHHHHH